(19) United States
(12) Patent Application Publication
(10) Pub. No.: US 2020/0030135 A1
(43) Pub. Date: Jan. 30, 2020

(54) MALE URINARY INCONTINENCE DEVICE

(71) Applicant: Charlton Woodyard, Washington, DC (US)

(72) Inventor: Charlton Woodyard, Washington, DC (US)

(21) Appl. No.: 16/513,375

(22) Filed: Jul. 16, 2019

Related U.S. Application Data
(60) Provisional application No. 62/702,854, filed on Jul. 24, 2018.

(51) Int. Cl. A61F 5/453; A61F 13/471

(52) U.S. Cl. CPC A61F 5/453; A61M 25/10; A61F 13/471

(57) ABSTRACT

Disclosed herein are male urinary incontinence devices, systems, and methods for urine collection. A male urinary incontinence device comprising: a sheathe sized to fit over the penis of a user comprising: a base, a shaft, and a head; a strap style retention member, wherein the strap style retention member is affixed to an undersurface of the sheathe further comprising: a left retention member strap having a refastenable securing means on the left retention member strap surface, a right retention member strap having the refastenable securing means on the right retention member strap surface; and a bottom portion adapted to fit over the sheathe at the distal end of the penis comprising: a lip portion wherein the lip portion is adapted to provide sealing adherence to the sheathe at the distal end of the penis, and an expandable balloon portion having an absorbent pad adapted to expand for collecting fluid.

### RELATED APPLICATION(S)

Under provisions of 35 U.S.C. § 119(e), the Applicant(s) claim the benefit of U.S. provisional application No. 62/702,854, filed Jul. 24, 2018, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### FIELD OF DISCLOSURE

The present disclosure relates to male urinary incontinence devices.

### BACKGROUND OF THE DISCLOSURE

Urinary incontinence is a significant health problem impacting between 3 to 11% of men according to recent United States National Institutes of Health Urology studies. Incontinence increases with age for men. Stress, trauma, prostate problems, neurological injury, and physical injury are among the typical causes of urinary incontinence. Thus, the conventional strategy is to medically address the underlying causes with treatments. While this may lead to long term recovery, the solutions offered don't effectively reduce the unintentional loss of urine. This often causes problems because the conventional strategy still allows embarrassment and poor management of urinary functions. For example, adult diapers, portable urinals, and urine collection containers still require extensive clean up and stain clothing. Accordingly, there remains a need for improved devices, methods, and systems for addressing urinary incontinence. This need and other needs are satisfied by the various aspects of the present disclosure.

### SUMMARY OF THE DISCLOSURE

A male urinary incontinence device may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

In accordance with the purposes of the disclosure, as embodied and broadly described herein, the disclosure, in one aspect, relates to a male urinary incontinence system having a sheathe configured to fit over the penis of a user having a base at the proximal end of the penis; a strap style retention member system adapted to secure the sheathe to a bottom portion hanging at the distal end of the penis using a refastenable securing means; and the bottom portion having an absorbent pad element, an expandable balloon portion, and a lip portion adapted to seal the bottom portion to the sheathe.

In yet another aspect, the disclosure relates to a method for managing male urinary incontinence comprising sliding a sheathe towards the proximal end of the penis of a user; affixing a strap style retention member radially over the sheathe at the proximal end of the penis of the user; securing the strap style retention member radially over the sheathe at the proximal end of the penis of the user using the refastenable securing component; attaching a bottom portion circumferentially around the sheathe; sealing the bottom portion to the sheathe by a lip portion of the bottom portion; and capturing excess fluid, using an absorbent pad component and expandable balloon component of the bottom portion.

In yet another aspect, the disclosure relates to a male urinary incontinence device comprising: a sheathe sized to fit over the penis of a user comprising: a base, a shaft, and a head; a strap style retention member, wherein the strap style retention member is affixed to an undersurface of the sheathe further comprising: a left retention member strap having a refastenable securing means on the left retention member strap surface, a right retention member strap having the refastenable securing means on the right retention member strap surface; and a bottom portion adapted to fit over the sheathe at the distal end of the penis comprising: a lip portion wherein the lip portion is adapted to provide sealing adherence to the sheathe at the distal end of the penis, and an expandable balloon portion having an absorbent pad adapted to expand for collecting fluid.

In further aspects, the disclosure relates to a male urinary incontinence device, such as, for example a sheathe, a penile sleeve, skin shield, or condom for urine collection. In further aspects, the sheathe, penile sleeve, skin shield, or condom may contain a non-return valve, seal, or lip to prevent the urine from leaking out of the male urinary incontinence device.

In another exemplary aspect, the disclosure relates to a male urinary incontinence device having a bulb shaped, or balloon shaped head at its tip.

In further aspects, the disclosure also relates to male urinary incontinence device having a bulb shaped, or balloon shaped head at its tip wherein the head may contain a reservoir, pouch, sleeve, or compartment for urine collection.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 shows a depiction of a male urinary incontinence device in accordance with an exemplary embodiment of the present invention.

FIG. 2 shows a depiction of a male urinary incontinence device in accordance with an exemplary embodiment of the present invention.

FIG. 3 shows a depiction of a male urinary incontinence device in accordance with an exemplary embodiment of the present invention.

FIG. 4A shows a depiction of a male urinary incontinence device in accordance with an exemplary embodiment of the present invention.

FIG. 4B shows a depiction of a male urinary incontinence device in accordance with an exemplary embodiment of the present invention.

FIG. 5 is a flow chart of a method for providing a male urinary incontinence device.

### DETAILED DESCRIPTION OF THE DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

As briefly described above, the present disclosure relates, in various aspects, to a male urinary incontinence device.

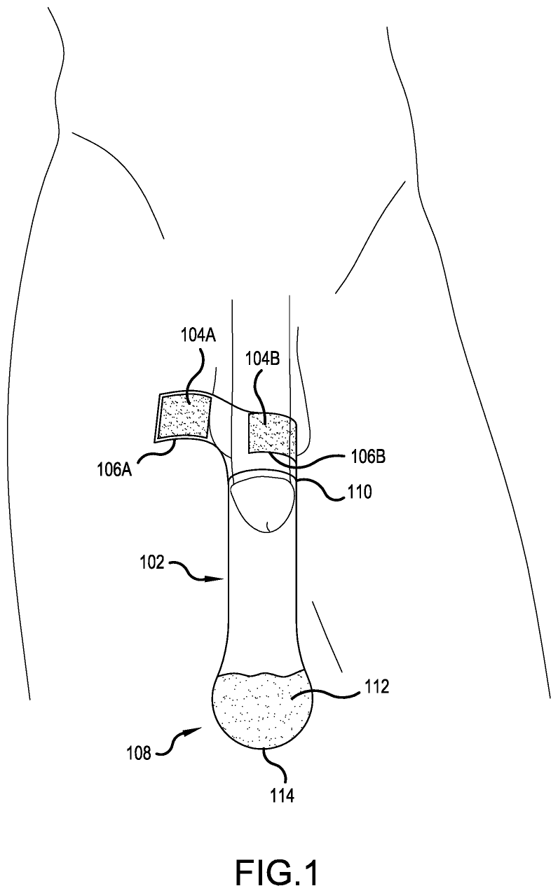

According to various further aspects of the disclosure, the male urinary incontinence devices and systems can comprise multiple configurations. For example, various exemplary embodiments of the inventive counting devices and systems are shown in the FIGS. 1-4B.

In aspects, FIGS. 1-4B show various views and features of an exemplary device for urine collection in accordance with the present disclosure. FIG. 1 shows a depiction of a male urinary incontinence device in accordance with an exemplary embodiment of the present invention. More specifically, FIG. 1 shows a front view of the male urinary incontinence device in accordance with an exemplary embodiment of the present invention. FIG. 1, 102 shows a sheathe over the penis of a user. FIG. 1, 106 shows a strap style retention member comprising: a left retention member strap 106A having a refastenable securing means on the left retention member strap surface 104A; and a right retention member strap 106B having a refastenable securing means on the right retention member strap surface 104B. FIG. 1, 108 showing a bottom portion 108 adapted to fit over the sheathe 102 at the distal end of the penis comprising a lip portion 110 wherein the lip portion 110 is adapted to provide sealing adherence to the sheathe 102 at the distal end of the penis, and an expandable balloon portion 114 having an absorbent pad 112 adapted to expand for collecting fluid. FIG. 1 shows the male urinary incontinence device with the refastenable securing means unfastened, in a filled state, meaning urine, fluid, or excess fluid dispensed from the penis has been collected in the male urinary incontinence device.

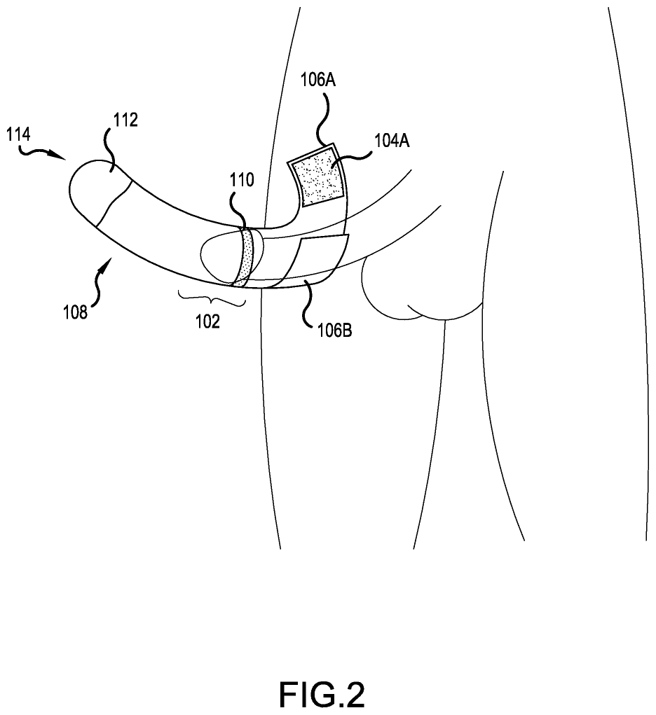

FIG. 2 shows a depiction of a male urinary incontinence device in accordance with an exemplary embodiment of the present invention. More specifically, FIG. 2 shows a side view of the male urinary incontinence device in accordance with an exemplary embodiment of the present invention. FIG. 2, 102 shows a sheathe over the penis of a user. FIG. 2, 106 shows a strap style retention member comprising: a left retention member strap 106A having a refastenable securing means on the left retention member strap surface 104A; and a right retention member strap 106B having a refastenable securing means on the right retention member strap surface 104B. FIG. 2, 108 showing a bottom portion 108 adapted to fit over the sheathe 102 at the distal end of the penis comprising a lip portion 110 wherein the lip portion 110 is adapted to provide sealing adherence to the sheathe 102 at the distal end of the penis, and an expandable balloon portion 114 having an absorbent pad 112 adapted to expand for collecting fluid. FIG. 2 shows the male urinary incontinence device with the refastenable securing means unfastened, in an empty state, meaning urine, fluid, or excess fluid dispensed from the penis has not been collected in the male urinary incontinence device.

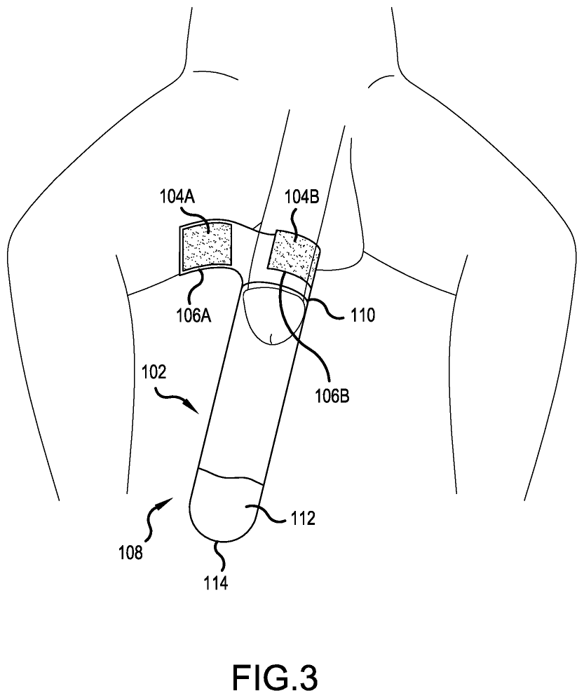

FIG. 3 shows a depiction of a male urinary incontinence device in accordance with an exemplary embodiment of the present invention. More specifically, FIG. 3 shows a top view of the male urinary incontinence device in accordance with an exemplary embodiment of the present invention. FIG. 3, 102 shows a sheathe over the penis of a user. FIG. 3, 106 shows a strap style retention member comprising: a left retention member strap 106A having a refastenable securing means on the left retention member strap surface 104A; and a right retention member strap 106B having a refastenable securing means on the right retention member strap surface 104B. FIG. 3, 108 showing a bottom portion 108 adapted to fit over the sheathe 102 at the distal end of the penis comprising a lip portion 110 wherein the lip portion 110 is adapted to provide sealing adherence to the sheathe 102 at the distal end of the penis, and an expandable balloon portion 114 having an absorbent pad 112 adapted to expand for collecting fluid. FIG. 3 shows the male urinary incontinence device with the refastenable securing means unfastened, in an empty state, meaning urine, fluid, or excess fluid dispensed from the penis has not been collected in the male urinary incontinence device.

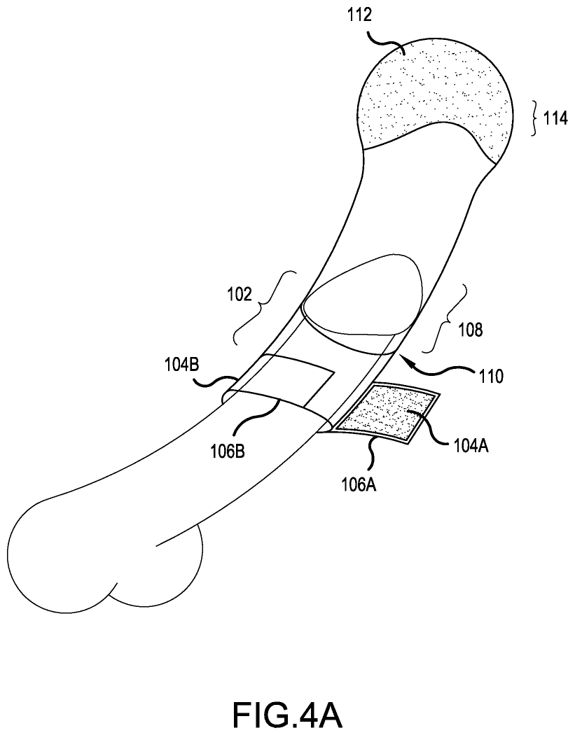

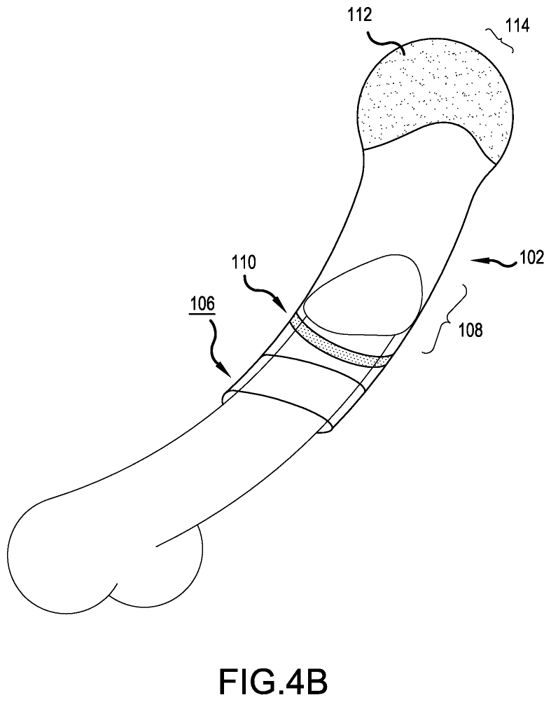

FIG. 4A shows a depiction of a male urinary incontinence device in accordance with an exemplary embodiment of the present invention. More specifically, FIG. 4A shows a perspective view of the male urinary incontinence device in accordance with an exemplary embodiment of the present invention. FIG. 4A, 102 shows a sheathe over the penis of a user. FIG. 4A, 106 shows a strap style retention member comprising: a left retention member strap 106A having a refastenable securing means on the left retention member strap surface 104A; and a right retention member strap 106B having a refastenable securing means on the right retention member strap surface 104B. FIG. 4A, 108 showing a bottom portion 108 adapted to fit over the sheathe 102 at the distal end of the penis comprising a lip portion 110 wherein the lip portion 110 is adapted to provide sealing adherence to the sheathe 102 at the distal end of the penis, and an expandable balloon portion 114 having an absorbent pad 112 adapted to expand for collecting fluid. FIG. 4A shows the male urinary incontinence device with the refastenable securing means unfastened, in an empty state, meaning urine, fluid, or excess fluid dispensed from the penis has not been collected in the male urinary incontinence device.

FIG. 4B shows a depiction of a male urinary incontinence device in accordance with an exemplary embodiment of the present invention. More specifically, FIG. 4B shows a perspective view of the male urinary incontinence device in accordance with an exemplary embodiment of the present invention. FIG. 4B, 102 shows a sheathe over the penis of a user. FIG. 4B, 106 shows a strap style retention member comprising: a left retention member strap 106A having a refastenable securing means on the left retention member strap surface 104A; and a right retention member strap 106B having a refastenable securing means on the right retention member strap surface 104B. FIG. 4B, 108 showing a bottom portion 108 adapted to fit over the sheathe 102 at the distal end of the penis comprising a lip portion 110 wherein the lip portion 110 is adapted to provide sealing adherence to the sheathe 102 at the distal end of the penis, and an expandable balloon portion 114 having an absorbent pad 112 adapted to expand for collecting fluid. FIG. 4B shows the male urinary incontinence device with the refastenable securing means fastened securing the strap style retention member. FIG. 4B shows the male urinary incontinence device in an empty state, meaning urine, fluid, or excess fluid dispensed from the penis has not been collected in the male urinary incontinence device.

In one or more embodiments, the lip portion 110 further comprises one or more interior ribs incorporated circumferentially around the interior of the bottom portion 108 affixed to the sheathe 102.

In one or more embodiments, the lip portion 110 further comprises one or more interior ribs incorporated circumferentially around the interior of the bottom portion 108 affixed to the sheathe 102. In one or more embodiments, the lip portion 110 may be affixed to at least one of a head of the sheathe 102, a shaft of the sheathe 102, and a base of the sheathe 102.

In one or more embodiments, the sheathe 102 may be at least one of: a penile sleeve, a skin shield, and a condom. In one or more embodiments, the male urinary incontinence device may further comprise wherein the refastenable securing means may be at least one of: a hook and loop fastener, a standard adhesive made of polypropylene, magic tape or mechanical tape, a button, and a snapping fastener.

In one or more embodiments, the male urinary incontinence device of may further comprise wherein the lip portion 110 may be adapted to provide sealing adherence to the sheathe 102 at the distal end of the penis. In further embodiments, the male urinary incontinence device may further comprise one or more rib elements configured to prevent fluid from flowing out of the bottom portion 108.

In these embodiments, a male urinary incontinence device is disclosed, comprising: a sheathe as shown in FIG. 1, 102 sized to fit over the penis of a user comprising a base, a shaft, and a head; a strap style retention member as shown in FIG. 1, 106, wherein the strap style retention member is affixed to an undersurface of the sheathe 102 further comprising a left retention member strap 106A having a refastenable securing means on the left retention member strap surface 104A, a right retention member strap 106B having the refastenable securing means on the right retention member strap surface 104B; and a bottom portion 108 adapted to fit over the sheathe 102 at the distal end of the penis comprising: a lip portion 110 wherein the lip portion 110 is adapted to provide sealing adherence to the sheathe at the distal end of the penis, and an expandable balloon portion 114 having an absorbent pad 112 adapted to expand for collecting fluid.

In further aspects, the male urinary incontinence device further comprises at least one of a penile sleeve, skin shield, sheathe, condom sheath, condom catheter, or condom for excess fluid collection.

In one or more embodiments, the male urinary incontinence device may be affixed to the distal end of the penis of a user. The male urinary incontinence device may be affixed right above the tip or head portion of the penis of a user. The strap style retention member of the male urinary incontinence device may be positioned at the distal end of the penis of a user. In one or more instances, the strap style retention member of the male urinary incontinence device may be positioned directly above the head or tip portion of the penis of the user.

In one or more embodiments, the sheath of the male urinary incontinence device may be hang vertically positioned below the tip or head portion of the penis of a user. The bottom portion, lip portion, and expandable balloon portion of the male urinary incontinence device may be positioned below the sheath of the male incontinence device. The bottom portion, lip portion, and expandable balloon portion of the male urinary incontinence device may hang lower depending on whether or not the male urinary incontinence device is in a used state, i.e. filled with fluids or in an empty state, i.e. no fluids are present.

In one or more embodiments, the male urinary incontinence device may comprise rigid materials including but not limited to plastic, latex, vinyl, rubber, and other materials.

In further aspects, the male urinary incontinence device further comprises at least one of a penile sleeve, skin shield, sheathe, condom sheath, condom catheter, or condom may further comprise a non-return valve, seal, or lip to prevent the urine from leaking out of the male urinary incontinence device.

In still further aspects, the disclosure relates to a male urinary incontinence device having a bulb shaped, or balloon shaped head at its tip.

In even further aspects, the disclosure also relates to male urinary incontinence device having a bulb shaped, or balloon shaped head at its tip wherein the head may contain a reservoir, pouch, sleeve, or compartment for urine collection.

In even further aspects, the male urinary incontinence device may be constructed from latex, silicone, polyurethane, polyisoprene, natural animal skin, non-porous materials, and hypoallergenic materials.

In still further aspects, the male urinary incontinence device comprising a non-return valve, seal, or lip may be configured to prevent the urine from leaking out of the male urinary incontinence device. The non-return valve, seal, or lip may be constructed from plastic, silicone, polyurethane, polyisoprene, natural animal skin, non-porous materials, and hypoallergenic materials.

In still further aspects, the male urinary incontinence device may further comprise, the head further including a compartment comprising of at least one of: a reservoir, a pouch, a sleeve, configured for urine collection.

In even further aspects, the male urinary incontinence device further comprising, a release mechanism for urine waste.

There is provided by the present disclosure, a system comprising: a male urinary incontinence device; an applicator; a release mechanism for urine waste.

In one or more embodiments, there is provided by the present disclosure, a system comprising: a sheathe configured to fit over the penis of a user having a base at the proximal end of the penis; a strap style retention member system adapted to secure the sheathe to a bottom portion hanging at the distal end of the penis using a refastenable securing means; and the bottom portion having an absorbent pad element, an expandable balloon portion, and a lip portion adapted to seal the bottom portion to the sheathe.

In still further aspects, the system may comprise a male urinary incontinence device; an applicator used on the male urinary incontinence device to simplify attachment to a user of the male urinary incontinence device; and a release mechanism for urine waste.

The present disclosure, according to further aspects, also provides methods of using the disclosed devices and systems. In one aspect, disclosed herein is a method for of using a male urinary incontinence device.

In further aspects, the disclosed male urinary incontinence devices and systems can be used for urine collection. In still further aspects, the disclosed male urinary incontinence devices and systems can be used to prevent the urine from leaking out of the male urinary incontinence device.

According to various further aspects of the disclosure, the male urinary incontinence devices and systems can comprise multiple configurations. For example, various exemplary embodiments of the inventive counting devices and systems are shown in the FIGS. 1-4B.

In aspects, FIGS. 1-4B show various views and features of an exemplary device for urine collection in accordance with the present disclosure. In these embodiments, the male urinary incontinence device comprises a sheathe, a penile sleeve, skin shield, condom sheath, condom catheter, or condom for urine collection.

In further aspects, the penile sleeve, skin shield, condom sheath, condom catheter, or condom for urine collection may contain a non-return valve, seal, or lip to prevent the urine from leaking out of the male urinary incontinence device.

In another exemplary aspect, the disclosure relates to a male urinary incontinence device having a bulb shaped, or balloon shaped head at its tip.

In further aspects, the disclosure also relates to male urinary incontinence device having a bulb shaped, or balloon shaped head at its tip wherein the head may contain a reservoir, pouch, sleeve, or compartment for urine collection.

In still further aspects, the present disclosure may be a male urinary incontinence device, comprising at least one of: a penile sleeve, skin shield, or condom configured for urine collection; a urine valve comprising at least one of: a non-return valve, seal, or lip; wherein the urine valve is configured to prevent a urine from leaking out of the male urinary incontinence device; a base; and a head.

In further aspects, the male urinary incontinence device further comprising, wherein the head further comprises at least one of: a bulb shape, a balloon shape, a shape at an apex of the head.

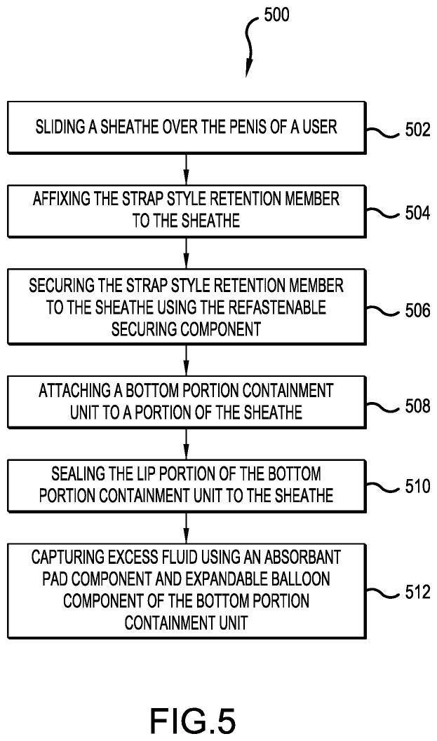

FIG. 5 is a flow chart setting forth the general stages involved in a method 500 consistent with an embodiment of the disclosure for providing a male urinary incontinence device.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 500 will be described in greater detail below.

Method 500 may begin at stage 502 where a user may slide a sheathe over the penis of a user. From stage 502, where the user slides the sheathe over the penis, method 500 may advance to stage 504 where the user may affix the strap style retention member to the sheathe.

Once the user affixes the strap style retention member to the sheathe in stage 504, method 500 may continue to stage 506 where a user may secure the strap style retention member to the sheathe using the refastenable securing component.

After a user secures the strap style retention member to the sheathe using the refastenable securing component in stage 506, method 500 may proceed to stage 508 where a user may attach a bottom portion containment unit to a portion of the sheathe. Once a user attaches a bottom portion containment unit to a portion of the sheathe in stage 508, method 500 may proceed to stage 510 where a user may seal the lip portion of the bottom portion containment unit to the sheathe.

After a user seals the lip portion of the bottom portion containment unit to the sheathe in stage 510, method 500 may proceed to stage 512 where the male incontinence device may capture excess fluid using an absorbent pad component and expandable balloon component of the bottom portion containment unit.

After the user the male incontinence device may capture excess fluid using an absorbent pad component and expandable balloon component of the bottom portion containment unit, method 500 may then end after stage 512.

In one or more embodiments, there is provided by the present disclosure a method for managing male urinary incontinence, the method comprising a step of sliding a sheathe towards the proximal end of the penis of a user. The method further including a next step of affixing a strap style retention member radially over the sheathe at the proximal end of the penis of the user. The method further comprising a step of securing the strap style retention member radially over the sheathe at the proximal end of the penis of the user using the refastenable securing component. The method further comprising a next step of attaching a bottom portion circumferentially around the sheathe. The method further comprising a next step of sealing the bottom portion to the sheathe by a lip portion of the bottom portion. The method further comprising a next step of capturing excess fluid, using an absorbent pad component and expandable balloon component of the bottom portion.

In further aspects, there is provided by the present disclosure, a method of using a male urinary incontinence device, the method comprising: at least one of: a penile sleeve, skin shield, or condom configured for urine collection; a urine valve comprising at least one of: a non-return valve, seal, or lip; wherein the urine valve is configured to prevent a urine from leaking out of the male urinary incontinence device; a base; and a head.

The following is claimed:

1. A male urinary incontinence device comprising:
a sheathe sized to fit over a penis of a user comprising:
a base, a shaft, and a head;
a strap style retention member, wherein the strap style retention member is affixed to an undersurface of the sheathe further comprising:
a left retention member strap having a refastenable securing means on the left retention member strap surface,
a right retention member strap having the refastenable securing means on the right retention member strap surface; and
a bottom portion adapted to fit over the sheathe at the distal end of the penis comprising:
a lip portion wherein the lip portion is adapted to provide sealing adherence to the sheathe at the distal end of the penis, and
an expandable balloon portion having an absorbent pad adapted to expand for collecting fluid.

2. The male urinary incontinence device of claim 1, wherein the lip portion further comprises one or more interior ribs incorporated circumferentially around an interior of the bottom portion affixed to the sheathe.

3. The male urinary incontinence device of claim 1 wherein the sheathe is at least one of: a penile sleeve, a skin shield, and a condom.

4. The male urinary incontinence device of claim 3 wherein the lip portion is attached to the head of the sheathe.

5. The male urinary incontinence device of claim 3 wherein the lip portion is attached to the shaft of the sheathe.

6. The male urinary incontinence device of claim 3, wherein the lip portion attached to the base of the sheathe.

7. The male urinary incontinence device of claim 1 wherein the refastenable securing means is at least one of: a hook and loop fastener, a standard adhesive made of polypropylene, magic tape or mechanical tape, a button, and a snapping fastener.

8. The male urinary incontinence device of claim 2 wherein the lip portion adapted to provide sealing adherence to the sheathe at the distal end of the penis further comprises: one or more rib elements configured to prevent fluid from flowing out of the bottom portion.

9. A male urinary incontinence system comprising:
a sheathe configured to fit over a penis of a user having a base at the proximal end of the penis;
a strap style retention member system adapted to secure the sheathe to a bottom portion hanging at the distal end of the penis using a refastenable securing means; and
the bottom portion having an absorbent pad element, an expandable balloon portion, and a lip portion adapted to seal the bottom portion to the sheathe.

10. A method for managing male urinary incontinence comprising:
sliding a sheathe towards the proximal end of a penis of a user;
affixing a strap style retention member radially over the sheathe at the proximal end of the penis of the user;
securing the strap style retention member radially over the sheathe at the proximal end of the penis of the user using a refastenable securing component;
attaching a bottom portion circumferentially around the sheathe;
sealing the bottom portion to the sheathe by a lip portion of the bottom portion; and
capturing excess fluid, using an absorbent pad component and expandable balloon component of the bottom portion.
